(19) United States
(12) Patent Application Publication
(10) Pub. No.: US 2015/0203806 A1
(43) Pub. Date: Jul. 23, 2015

(54) SYSTEMS FOR DISRUPTING BIOLOGICAL SAMPLES AND ASSOCIATED DEVICES AND METHODS

(71) Applicant: UW Center for Commercialization, Seattle, WA (US)

(72) Inventors: Paul Yager, Seattle, WA (US); Peter C. Kauffman, Bainbridge Island, WA (US); Joshua Buser, Seattle, WA (US); Samantha Byrnes, Seattle, WA (US); Alec K. Wollen, Seattle, WA (US); Erin Heiniger, Seattle, WA (US)

(21) Appl. No.: 14/601,966

(22) Filed: Jan. 21, 2015

Related U.S. Application Data
(60) Provisional application No. 61/929,769, filed on Jan. 21, 2014.

(51) Int. Cl. C12M 1/00; C12M 1/42; C12N 13/00

(52) U.S. Cl. CPC C12M 47/06; C12N 13/00; C12M 35/06

(57) ABSTRACT

The present technology relates generally to systems for disrupting biological samples and associated devices and methods. In some embodiments, system includes a vessel configured to receive the biological sample, a permanent magnet configured to be positioned within the vessel, an electromagnet configured to be positioned proximate the vessel, and a current source operably coupled to the electromagnet and configured to transmit an alternating current. In some embodiments, when the biological sample is placed within the vessel and the alternating current is transmitted to the electromagnet, the electromagnet produces an alternating magnetic field that causes the permanent magnet to rotate within the vessel, thereby lysing at least one of the cells of the biological sample.

### CROSS-REFERENCE TO RELATED APPLICATION(S)

This application claims the benefit of U.S. Provisional Application No. 61/929,769, filed Jan. 21, 2014, titled “Electromechanical Cell Lysis Using a Mobile Electronic Device,” which is incorporated herein by reference in its entirety.

### STATEMENT REGARDING FEDERALLY SPONSORED RESEARCH

This invention was made with U.S. government support under HR0011-11-2-0007, awarded by the Defense Advanced Research Projects Agency. The U.S. Government has certain rights in the invention.

### TECHNICAL FIELD

The present technology relates generally to systems for disrupting biological samples and associated devices and methods. Many embodiments of the present technology relate to systems for lysing cells and associated devices and methods.

### BACKGROUND

Diagnosis is the first hurdle in disease management, enabling expedited appropriate treatment in developed settings where sophisticated equipment and trained personnel are available. For example, in the United States, in-vitro diagnostic procedures represent about 1.6% of Medicare spending, yet influence 60-70% of medical decisions. Nucleic acid amplification tests (NAATs) performed in the laboratory represent the pinnacle of sensitive and specific pathogen detection. Unfortunately, this state of the art is also expensive and complex, requiring infrastructure and instrumentation not available in all settings.

The lack of adequate diagnostics is especially troublesome in the case of tuberculosis (TB), which infects approximately one-third of the world's population according to the World Health Organization (WHO). Sixty percent of TB patients only have access to a peripheral level of the health system, where no suitable TB diagnostics exist. Conventional TB diagnostics in low-resource settings, mainly sputum smear microscopy and cell culture, lack the ideal specificity and timeliness. Also, the required equipment is rarely available.

Microfluidic devices have shown promise to enable the type of point-of-care device that could bring NAATs to the point of care in low-resource settings, but sample preparation, such as cell lysis, remains the weak link in microfluidics-based bioassays. Mechanical lysis methods, such as bead beating, are desirable in that one can avoid the need to purify the sample from a chemical lytic agent before the downstream bioassay, but these methods traditionally suffer from relatively complex, user- and power-intensive instruments and protocols.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a partial cross-sectional front view of a lysis system configured in accordance with an embodiment of the present technology.

FIG. 2 is a graphical representation showing the effect of bead mass on lysing efficiency.

FIG. 3 is a front cross-sectional view of another lysis system configured in accordance with the present technology.

### DETAILED DESCRIPTION

The present technology is generally related systems for disrupting biological samples and associated devices and methods. A system for disrupting biological samples includes a vessel configured to receive the biological sample, a permanent magnet configured to be positioned within the vessel. An electromagnet is configured to be positioned proximate the vessel and a current source is operably coupled to the electromagnet and configured to transmit an alternating current. In some embodiments, when the biological sample is placed within the vessel and the alternating current is transmitted to the electromagnet, the electromagnet produces an alternating magnetic field that causes the permanent magnet to rotate within the vessel, thereby lysing at least one of the cells of the biological sample.

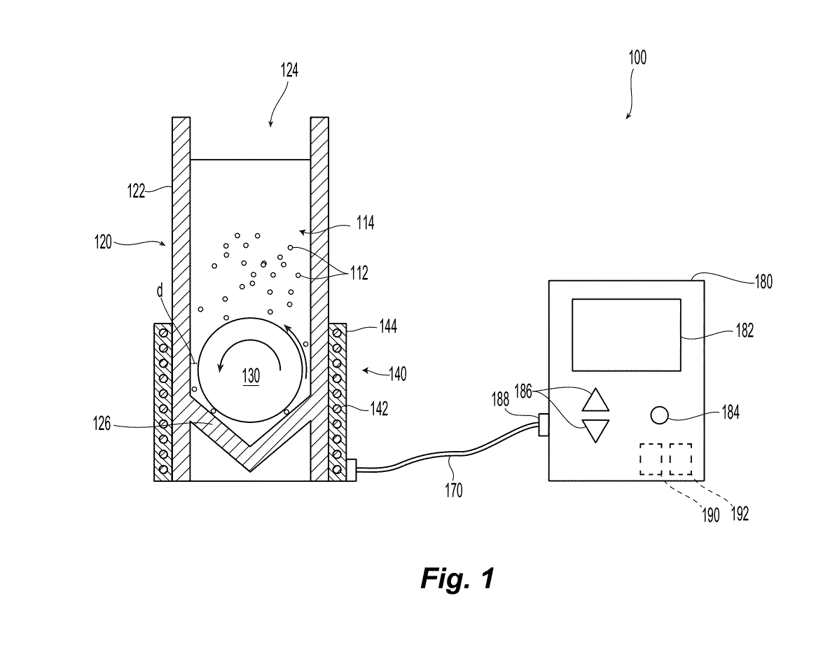

Specific details of several embodiments of the present technology are described below with reference to FIGS. 1-3. Although many of the embodiments are described below with respect to devices, systems, and methods for lysing cells, other embodiments are within the scope of the present technology. For example, devices, systems, and methods of the present technology can be used to disrupt (e.g., mechanically, electrically, and/or chemically) or agitate any non-cellular biological sample (e.g., mucus) and/or non-cellular components of the biological sample. Additionally, other embodiments of the present technology can have different configurations, components, and/or procedures than those described herein. For example, other embodiments can include additional elements and features beyond those described herein, or other embodiments may not include several of the elements and features shown and described herein.

FIG. 1 is a partial cross-sectional front view of an assembled lysis system 100 (also referred to herein as “system 100”) configured in accordance with an embodiment of the present technology. The system 100 of FIG. 1 is shown during a lysis procedure. The system 100, for example, is configured to lyse one or more cells of a biological sample 114. As used herein, a “biological sample” can be any solid or fluid sample, living or dead, obtained from, excreted by, or secreted by any living or dead organism, including, without limitation, single-celled organisms, such as bacteria, yeast, protozoans, amoebas, multicellular organisms (such as plants or animals, including samples from a healthy or apparently healthy human subject or a human patient affected by a condition or disease to be diagnosed or investigated, such as tuberculosis) and/or soil. Biological samples can include one or more cells, proteins, nucleic acids, etc., as well as one or more buffers. Biological samples can be a liquid phase solution of cells or it may be a solid cell sample such as a cell pellet derived from a centrifugation procedure. As used herein, a “cell” or “cells” can refer to eukaryotic cells, prokaryotic cells, viruses, endospores or any combination thereof. Cells thus may include bacteria, bacterial spores, fungi, virus particles, single-celled eukaryotic organisms (e.g., protozoans, yeast, etc.), isolated or aggregated cells from multi-cellular organisms (e.g., primary cells, cultured cells, tissues, whole organisms, etc.), or any combination thereof, among others. Furthermore, the term “lysis” or “lyse” as used herein refers to disrupting the structural integrity of a cell (e.g., by breaking the cellular membrane of the cell) in order to gain access to materials within the cell.

As shown in FIG. 1, the lysis system 100 can include a vessel 120, a permanent magnet 130, an electromagnet 140, and a current source 180 operably coupled to the electromagnet 140 (e.g., via a cable 170). As discussed in greater detail below, when the biological sample 114 and permanent magnet 130 are placed within the vessel 120 and the current source 180 is activated, the electromagnet 142 produces an alternating magnetic field that causes the permanent magnet 130 to rotate within the vessel 120, thereby lysing at least one of the cells of the biological sample 114.

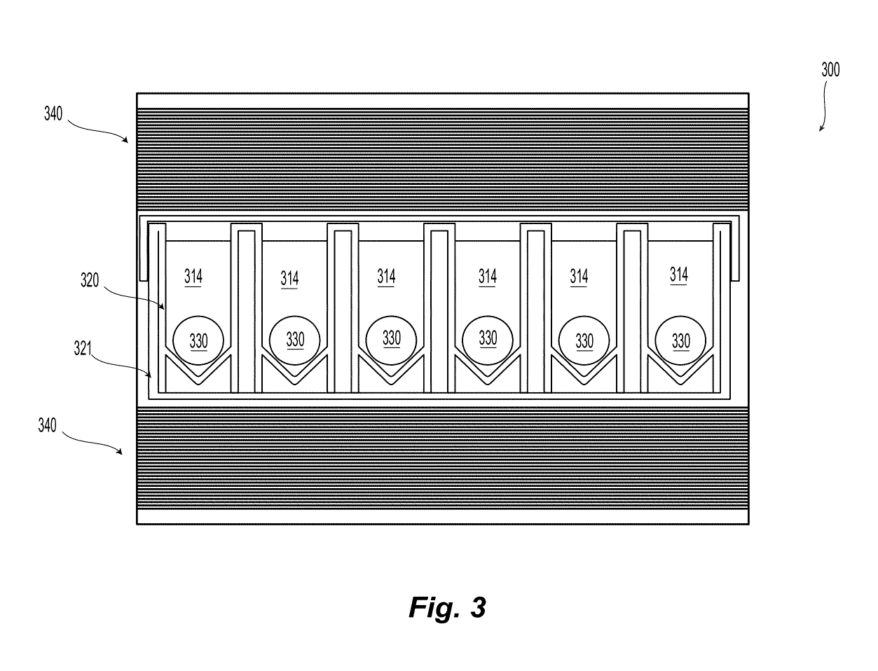

The vessel 120 can be a tube (e.g., a laboratory tube) having a generally cylindrical sidewall 122 and a conical bottom portion 126 that together define an interior portion of the vessel 120. The vessel 120 may, for example, be in the shape of a micro centrifuge tube (e.g., an Eppendorf tube), a centrifuge tube, a vial, etc. As shown in FIG. 1, the vessel 120 can define only one compartment/chamber for holding the biological sample 114, or a plurality of discrete compartments/chambers (e.g., an array of wells) for holding biological samples in isolation from one another (e.g., a microwell plate, discussed in greater detail below with reference to FIG. 3). The vessel 120 can include an opening 124 at a top portion for delivering a biological sample to the interior portion of the vessel 120. In those embodiments where the vessel 120 remains vertically-oriented (as shown in FIG. 1), the vessel 120 can remain generally open to the environment during a lysis procedure. In other embodiments, however, the vessel 120 can include a cap (not shown) and can be configured to be sealed before, during, and/or after the lysis procedure. The vessel 120 can be made of plastic and/or other suitable materials.

It will be appreciated that although the vessel 120 shown in FIG. 1 has a generally tubular shape with a conical bottom portion 126, in other embodiments, the vessel 120 and/or any portion of the vessel 120 can have any suitable size or shape, and/or be made of any suitable material. For example, in some embodiments the vessel 120 can have a rounded bottom portion 126 (not shown). In a particular embodiment, the vessel 120 can have a bottom portion 126 configured to mirror the shape of the permanent magnet 130. For example, in those embodiments where the system 100 includes a spherical permanent magnet 130 (as in FIG. 1), the shape of the bottom portion 126 can follow the shape of the spherical permanent magnet (e.g., the vessel 120 can be shaped like a narrow- or wide-necked round-bottom flask).

The system 100 can optionally include a plurality of beads 112. The beads 112 may be pre-loaded into the vessel 120, or the user may add the beads 112 during the lysis procedure. The beads 112 can be any particle-like and/or bead-like structure that has a hardness greater than the hardness of the cells targeted for lysis. The beads 112 may be made of plastic, glass, ceramic, metal and/or any other suitable materials. In certain embodiments, the beads 112 may be made of non-magnetic materials. The beads 112 can be rotationally symmetric about at least one axis (e.g., spherical, rounded, oval, elliptic, egg-shaped, droplet-shaped particles, etc.). In other embodiments, however, the beads 112 can have a polyhedron shape. In some embodiments, the beads 112 can be irregularly-shaped particles and/or include protrusions. In certain embodiments, the mass of beads 112 added to the vessel 120 can be between about 1 mg to about 10,000 mg, about 1 mg to about 1000 mg, about 1 mg to about 100 mg, and about 1 mg to about 10 mg, etc. In a particular embodiment, the bead mass can be between about 200 mg to about 400 mg. In certain embodiments, the individual beads 112 can have a diameter in the range of about 10 μm to about 1,000 μm, about 20 μm to about 400 μm, or about 50 μm to about 200 μm. The system 100 can include beads 112 having the same or different diameters.

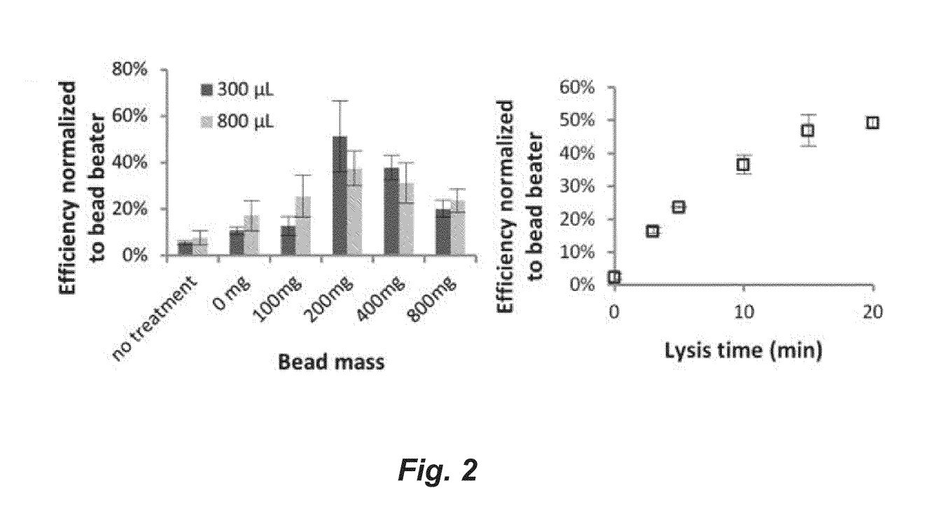

Without being bound by theory, it is believed that the size and mass of beads 112 present in the vessel 120 during the lysis procedure affects the viscosity of the lysing medium, which affects the lysing efficiency. Lysing efficiency can be quantified, for example, by measuring the amount of DNA recovered from the lysed sample (e.g., via quantitative nucleic acid amplification). As demonstrated by the graphs shown in FIG. 2, up to a certain mass, the addition of beads 112 to the biological sample 114 during the lysis procedure can increase the lysing efficiency of the system 100. However, it is also believed that, once the beads 112 reach a critical mass, the beads 112 can decrease the lysing efficiency. As such, it will be appreciated that the size and mass of the beads 112 added to the biological sample 114 can be varied based on the selected biological sample.

Referring back to FIG. 1, the permanent magnet 130 may be pre-loaded into the vessel 120, or the user (not shown) may add the permanent magnet 130 during the lysis procedure. The permanent magnet 130 can be generally spherical and configured to be positioned within the vessel 120 adjacent a bottom portion 126 of the vessel 120. In other embodiments, the permanent magnet 130 can have other suitable shapes. For example, in some embodiments, the permanent magnet 130 can be generally cylindrical, disc-shaped, cubical, and/or other suitable polyhedrons and non-polyhedrons. The permanent magnet 130 can be made from a material that is magnetized and creates its own persistent magnetic field. For example, the permanent magnet 130 can be made from iron, nickel, cobalt, rare-earth metals and some of their alloys (e.g., an Alnico magnet, a neodymium magnet, etc.), naturally occurring minerals such as lodestone, and other suitable materials. As shown in FIG. 1, the permanent magnet 130 can have a diameter that is slightly less than the inner diameter of the vessel 120 such that an outer surface of the permanent magnet 130 is separated from the inner surface of the vessel 120 by a small distance d. The distance d can be small enough to create a region of high shear between the permanent magnet 130 and the interior surface of the vessel 120 when the permanent magnet 130 rotates, but large enough to allow the permanent magnet 130 to rotate freely about any of its plurality of axes, as well as to provide passage for the beads 112 and/or cells during rotation of the magnet 130. In other embodiments, the permanent magnet 130 can have other suitable sizes, and/or the system 100 can include more than one permanent magnet 130 (e.g., two permanents magnets, three permanent magnets, etc.).

In some embodiments, the inner surface of the vessel 120 and/or the outer surface of the permanent magnet 130 may include one or more protrusions (not shown) or may be otherwise texturized to increase the surface area of the respective surface and improve lysing efficiency. For example, one or more protrusions can be adhered to or formed on the outer surface of the permanent magnet 130 and/or inner surface of the vessel 120 (e.g., via adhesive, soldering, welding, electrodeposition, etc.). The protrusions can have any suitable shape, size and/or configuration (e.g,. spherical, cubical, cylindrical, half-spherical, polyhedron, non-polyhedron, etc.).

The electromagnet 140 includes a coiled magnet wire 142 embedded within or surrounded by a tubular support 144. In other embodiments, the magnet wire is in a spiral or helical configuration. In some embodiments, the electromagnet 140 only includes the magnet wire 142 (and not the support 144). The electromagnet 140 is configured to be electrically coupled to the current source 180 (e.g., via a cable 170). When activated, the current source 180 delivers an alternating current (e.g., an electrical audio signal) to the magnet wire 142 of the electromagnet 140.

The current source 180 can be a battery-powered portable electronic device (e.g., a mobile electronic device) capable of generating an electrical audio signal. For example, the current source 180 can be configured to generate an alternating current that alternates between about 10 Hz and about 90 Hz. In some embodiments, the current source 180 can generate an alternating current that alternates between about 20 Hz and about 60 Hz (e.g., about or equal to 30 Hz, about or equal to 40 Hz, about or equal to 60 Hz, etc.). The current source 180 can include a cell phone, a portable audio device (e.g., a portable mp3 player, a portable radio, a portable cd player, a tape player, etc.), a tablet, a laptop, or other suitable devices. In some embodiments, the current source 180 can include a display screen 182, an electrical output 188 (e.g., an audio jack), and one or more controls. In some embodiments, the display screen 182 is a touch screen. The display screen 182 can indicate to the user various signal parameters, such as the time elapsed, the frequency at which the current is alternating, and the waveform. The current source 180 can further include a power button 184 and optional control buttons 186 to adjust one or more of the parameters. In some embodiments, the control buttons 186 may be incorporated into a touch-screen display.

The current source 180 can further include a processor 190 and memory 192. The memory 192 can include one or more programs. Each of the programs can include one or more pre-set signal parameters. For example, a first program can output a 30 Hz signal with a sinusoidal waveform, and a second program can output a 40 Hz signal with a square wave waveform. The programs need not have different values for each parameter. In some embodiments, each of the programs can be tailored to a different lysis procedure. For example, lysis of stronger cells, such as mycobacterium tuberculosis (MTB), may require a higher frequency and/or a longer duration of agitation. As such, the current source 180 may contain a program specifically designed for lysis of MTB cells that includes a relatively higher frequency. In some embodiments, one or more programs can be downloaded to the current source 180 via a hard connection or wirelessly. For example, a frequency and waveform generator application, such as FreqGen (William Ames), can be downloaded to the current source 180 and supply a variety of waveforms at a wide range of frequencies. In some embodiments, the system 100 can further include an amplifier (not shown) to increase the power delivered by the current source 180.

A method for using the system 100 disclosed herein will now be described with reference to FIG. 1. In those embodiments of the system 100 where the beads 112 and/or the permanent magnet 130 are not pre-loaded into the vessel 120, the method can include delivering one or more of the beads 112 and the permanent magnet 130 to the vessel 120. The method further includes delivering the biological sample 114 to the vessel 120. The beads 11, the biological sample 114 (referred to collectively herein as the “lysing mixture”), and the permanent magnet 130 can be delivered to the vessel 120 in any order.

Before or after delivering the lysing mixture to the vessel 120, the vessel 120 can be positioned within an operating distance of the electromagnet 140. As used herein, “operating distance” refers to a distance between the electromagnet 140 and the permanent magnet 130 whereby an alternating current traveling through the electromagnet 140 causes the permanent magnet 130 to move. As shown in FIG. 1, in some embodiments, the vessel 120 can be positioned within the core of the electromagnet 142. In such embodiments, the electromagnet 140 can be positioned around a portion of the vessel 120 corresponding to the location of the permanent magnet 130. For example, the electromagnet 140 and/or vessel 120 can be positioned such that a top-most portion of the permanent magnet 130 is positioned at an elevation that is aligned with or below an elevation of the top-most turn of the magnet wire 142. In other embodiments, the electromagnet 140 and/or vessel 120 can be positioned such that a top-most portion of the permanent magnet 130 is positioned anywhere along the height of the vessel 120 and/or permanent magnet 130. In yet other embodiments, the electromagnet 140 can be positioned in other suitable locations relative to the vessel 120 and within an operating distance of the permanent magnet 130, such as below the vessel 120, to the side of the vessel 120, above the vessel 120, etc.

Upon positioning the vessel 120 and electromagnet 140, the user can then activate the current source 180 to deliver an alternating current to the wire 142 of the electromagnet 140. The vessel 120 and/or electromagnet 140 can be moved relative to one another at any point during the activation of the electromagnet 140. As the alternating current passes through the magnet wire 142, the direction of the magnetic field continuously alternates. In response to the alternating magnetic field caused by the electromagnet 140, the permanent magnet 130 is alternatingly attracted to and repelled from the electromagnet 140, thereby causing the permanent magnet 130 to rotate. In some embodiments, the permanent magnet 130 can rotate about its central axis such that the center of mass of the permanent magnet 130 remains substantially stationary. In other embodiments, the permanent magnet's 130 center of mass moves while it rotates (e.g., the permanent magnet 130 “bounces around” while it rotates). In such embodiments, the permanent magnet 130 may collide with the vessel wall.

Rotation of the permanent magnet 130 within the vessel 120 creates a region of high shear stress between the permanent magnet 130 and the interior surface of the vessel 120. The permanent magnet's 130 rotation also causes the lysing mixture to travel around at least a portion of the permanent magnet 130 and through the high shear regions. When traveling through these high shear regions, the cells encounter one or more destructive forces, such as shear stress and forces associated with collisions with the permanent magnet 130, beads 112 and/or the vessel 120. As such, over time one or more cells of the biological sample lyse.

FIG. 3 is a front view cross-sectional view of a portion of another system 300 configured in accordance with the present technology (a current source is not shown for ease of illustration). The system 300 can be generally similar to the system 100 described with reference to FIG. 1, except the system 300 includes two electromagnets 340 and a plurality of vessels 320 (only one labeled for ease of illustration) coupled by a support 321. In other embodiments, the system 300 can include a single electromagnet 340 or more than two electromagnets 340 (e.g., three electromagnets, four electromagnets, etc.). As shown in FIG. 3, the electromagnets 340 can be positioned at opposite ends of the support 321. In other embodiments, the electromagnets 340 can have other suitable configurations. Methods for using the system 300 for lysing cells can be generally similar to the methods described herein for use of the system 100.

The lysis systems disclosed herein can include additional features to improve lysing efficiency. For example, in some embodiments, the lysis systems disclosed herein can include a temperature control device. Additionally, in some embodiments, the lysis systems can include one or more feedback mechanisms. For example, in some embodiments the system 100 can include a current source having an electrical input jack (e.g., a microphone jack), an additional cable, and a microphone (e.g., a magnetic coil coupled to a diaphragm). During the lysis procedure, the cable can be coupled to the current source (e.g., via the input jack) and the microphone, and the microphone can be positioned adjacent the vessel 120 and/or electromagnet 140. Rotation of the permanent magnet 130 creates an electromagnetic field that can be monitored by the microphone and processed by the current source. For example, the additional voltage can be superimposed on the input signal which can be monitored by the current source to determine the voltage, frequency, and/or waveform of the superimposed signal. Abnormal changes in voltage can be detected by comparing the input voltage to a calibration curve (e.g., developed by manually spinning a magnet inside the coil of wire and measuring the voltage waveform). In some embodiments, the microphone can additionally or alternatively monitor the acoustic signature (frequencies, waveform) of the permanent magnet hitting the tube as it rotates.

Lysis systems configured in accordance with embodiments of the present technology provide several advantages over conventional mechanical lysis devices. First, the lysis system of the present technology achieves cell lysis with relatively inexpensive materials and at a significantly lower cost to the user. Second, the lysis system of the present technology is self-powered, and thus does not require an electrical outlet. Moreover, the lysis system disclosed herein consumes very little power, and thus (1) can operate for extended periods of time without needing to re-charge, and (2) can operate at lower temperatures (as compared to conventional devices), which can be beneficial for avoiding damage to any RNA and/or proteins that may be present in the biological sample.

The various embodiments described above can be combined to provide further embodiments. The embodiments, features, systems, devices, materials, methods, and techniques described herein may, in some embodiments, be similar to any one or more of the embodiments, features, systems, devices, materials, methods, and techniques described in U.S. Provisional Patent Application No. 61/289,156, filed Dec. 22, 2009, PCT Application No. PCT/US2010/061675, filed Dec. 21, 2010, U.S. Provisional Patent Application No. 61/501,055, filed Jun. 24, 2011, PCT Application No. PCT/US2012/044060, filed Jun. 25, 2012, U.S. patent application Ser. No. 13/518,365, filed Jun. 21, 2012, U.S. patent application Ser. No. 14/129,078, filed Mar. 24, 2014, U.S. Provisional Application No. 61/832,356, filed Jun. 7, 2013, U.S. Provisional Patent Application No. 61/861,055, filed Aug. 1, 2013, PCT Application No. PCT/US2014/012618, filed Jan. 22, 2014, U.S. Provisional Patent Application No. 61/929,769, filed Jan. 21, 2014, U.S. Provisional Patent Application No. 61/808,106, filed Apr. 3, 2013, U.S. Provisional Patent Application No. 61/832,536, filed Jun. 7, 2013, U.S. Provisional Patent Application No. 61/868,006, filed Aug. 20, 2013, and U.S. Provisional Patent Application No. 61/867,950, filed Aug. 20, 2013, all of which are incorporated by reference in their entireties. Aspects of the disclosed embodiments can be modified, if necessary, to employ concepts of the various patents, applications, and publications to provide yet further embodiments. For example, in some embodiments, the system may include a vessel having a first end, a second end opposite the first end, and a permanent magnet positioned therebetween. The first end of the vessel can be configured to receive one or more biological samples, and the second end of the vessel can be configured to be positioned in fluid communication with one or more of the microfluidic devices and systems detailed in one or more of the patent applications listed above. For example, the second end may be open such that the biological sample passes through the portion of the vessel housing the permanent magnet and exits into engagement with the microfluidic device or system. In some embodiments, the second end can include a filter, valve, or other device spanning at least a portion of the inner diameter of the vessel at the second end. When the current source of the system is activated, one or more cells of the biological sample may be lysed while passing through the portion of the vessel housing the permanent magnet.

From the foregoing, it will be appreciated that specific embodiments of the invention have been described herein for purposes of illustration, but well-known structures and functions have not been shown or described in detail to avoid unnecessarily obscuring the description of at least some embodiments of the invention. The systems described herein can perform a wide range of processes for preparing biological specimens for analyzing. Where the context permits, singular or plural terms may also include the plural or singular term, respectively. Unless the word “or” is associated with an express clause indicating that the word should be limited to mean only a single item exclusive from the other items in reference to a list of two or more items, then the use of “or” in such a list shall be interpreted as including (a) any single item in the list, (b) all of the items in the list, or (c) any combination of the items in the list. The singular forms “a,” “an,” and “the” include plural referents unless the context clearly indicates otherwise. Thus, for example, reference to “a specimen” refers to one or more specimens, such as two or more specimens, three or more specimens, or four or more specimens.

I/We claim:

1. A system, comprising:
a vessel configured to receive a biological sample;
a permanent magnet configured to be positioned within the vessel;
an electromagnetic coil configured to be positioned proximate the vessel; and
a current source configured to transmit an alternating current, wherein the current source is configured to be operably coupled to the electromagnet,
wherein, when the biological sample is placed within the vessel and the alternating current is transmitted to the electromagnet, the electromagnet produces an alternating magnetic field that causes the permanent magnet to rotate within the vessel, thereby disrupting at least a portion of the biological sample.

2. A system for lysing one or more cells of a biological sample, the system comprising:
a vessel configured to receive the biological sample;
a permanent magnet configured to be positioned within the vessel;
an electromagnetic coil configured to be positioned proximate the vessel; and
a current source configured to transmit an alternating current, wherein the current source is configured to be operably coupled to the electromagnet,
wherein, when the biological sample is placed within the vessel and the alternating current is transmitted to the electromagnet, the electromagnet produces an alternating magnetic field that causes the permanent magnet to rotate within the vessel, thereby lysing at least one of the cells of the biological sample.

3. The system of claim 2 wherein the permanent magnet is configured to rotate about any of its plurality of axes when positioned within an operating distance of the alternating magnetic field of the electromagnet.

4. The system of claim 2 wherein the permanent magnet is configured to be positioned within an end portion of the vessel, and wherein the electromagnet is configured to be positioned around a full circumference of the end portion.

5. The system of claim 2 wherein, when the permanent magnet is positioned within the vessel, the electromagnet surrounds a full circumference of the magnet.

6. The system of claim 2 wherein the current source is a mobile electronic device.

7. The system of claim 2 wherein the current source is an audio jack of a mobile electronic device.

8. The system of claim 2 wherein the current source is configured to be powered by a battery.

9. The system of claim 2 wherein the system comprises a single electromagnet.

10. The system of claim 2 wherein the vessel is generally tubular.

11. The system of claim 2 wherein the vessel has a first end and a second end opposite the first end, and wherein the second end is conical.

12. The system of claim 2 wherein the vessel has a first end and a second end opposite the first end, and wherein the second end is rounded.

13. The system of claim 2 wherein the permanent magnet is spherically-shaped.

14. The system of claim 2 wherein the permanent magnet is disk-shaped.

15. The system of claim 2, further comprising a plurality of beads configured to be delivered to the vessel before the alternating current is transmitted to the electromagnet.

16. The system of claim 2, further comprising a plurality of lysis aids configured to be delivered to the vessel before the alternating current is transmitted to the electromagnet.

17. A method of lysing cells in a biological sample, the method comprising:
delivering a biological sample to a vessel having a permanent magnet positioned therein;
positioning the vessel adjacent an electromagnet;
applying an alternating current to the electromagnet, thereby causing the permanent magnet to rotate; and
as the permanent magnet rotates about any of its plurality of axes, lysing at least one of the cells in the biological sample.

18. The method of claim 17 wherein positioning the vessel adjacent the electromagnet comprises positioning the vessel adjacent a single electromagnet.

19. The method of claim 17 wherein the permanent magnet is positioned within an end portion of the vessel, and wherein positioning the vessel adjacent the electromagnet further comprises positioning the electromagnet around the end portion.

20. The method of claim 17 wherein the applied alternating current alternates between about 10 Hz and about 60 Hz.

21. The method of claim 17 wherein the method does not include delivering a plurality of beads to the vessel.

22. The method of claim 17 wherein applying the alternating current comprises activating a current source, and wherein the current source is a mobile electronic device.

23. The method of claim 17, further comprising positioning the permanent magnet within the vessel before delivering the biological sample.
